(19) United States
(12) Patent Application Publication
(10) Pub. No.: US 2019/0147972 A1
(43) Pub. Date: May 16, 2019

(54) BIOMOLECULE DESIGN MODEL AND USES THEREOF

(71) Applicant: University of Notre Dame du Lac, South Bend, IN (US)

(72) Inventors: Brian M. Baker, Granger, IN (US); Tim Riley, South Bend, IN (US); Cory Ayres, MIshawaka, IN (US)

(73) Assignee: University of Notre Dame du Lac, South Bend, IN (US)

(21) Appl. No.: 16/090,766

(22) PCT Filed: Mar. 24, 2017
(86) PCT No.: PCT/US17/24154

§ 371 Date: Oct. 2, 2018

Related U.S. Application Data
(60) Provisional application No. 62/312,762, filed on Mar. 24, 2016.

(51) Int. Cl. G16B 5/20; C07K 14/725

(52) U.S. Cl. CPC G16B 5/20; C07K 14/7051

(57) ABSTRACT

Disclosed are computational modeling methods employing RMS fluctuation values associated with energy functions to compute binding properties of a subject biomolecule and an identified target. An energy function (or force field) that relates the molecular structure of a biomolecule to an energy value, modified with terms calculated from sets of RMS fluctuation values of the biomolecule, the target and the complex, are used to identify a potential mutation or modification suitable for imparting a selected property to a biomolecule of interest. Uses of the method in the manufacture of non-native proteins having a selected modified property are also provided. Therapeutic agents (proteins, antibodies, TCRs) enzymes, etc., prepared according to the present methods are also provided. Non-native biomolecules having improved properties, for example, weaker or enhanced binding affinity in a modified TCR, are described. Enzymes, industrial reagents, and the like, created using the disclosed methods are also presented.

### GOVERNMENT SUPPORT

The invention was made with U.S. government support under grant number GM103773, GM067079, GM075762 and UL1TR001108 from the National Institutes of Health. The U.S. government owns certain rights in the invention.

### TECHNICAL FIELD

The present invention relates to the field of computational methods for biomolecule design, as well as to improved biomolecules for therapeutic, industrial and other applications.

### BACKGROUND ART

Molecular recognition underlies all biological processes through interaction of biomolecules (often proteins, generally called receptors) with other proteins, peptides, or other molecules (also generally called ligands). This molecular recognition process involves changes in conformational degrees of freedom not only for receptors but also for the ligands. When any two molecules interact, binding can induce or facilitate a change in conformation for both molecules. For instance, when a ligand binds to a protein, a conformational change can occur in both the ligand and the protein. Similarly, when a protein binds to another protein, conformation changes can occur in both proteins.

Docking is a method for predicting binding locations, orientations and conformations of molecules when they interact to form a complex. The docking process can be used, for instance, in rational drug design, where design of one molecule (generally the drug) is based on knowledge of a target molecule. One important area where docking is important is in the immune system. For example, with antibodies or T-cell receptors.

Conformational changes in biomolecules and targets that occur when they interact are facilitated by the molecular flexibility of the biomolecule and its target.

Evaluation of potential conformations of a particular molecule can depend on, for instance, the interaction energy between the two molecules for each potential conformation of the particular molecule where potential conformations are dictated by flexibility.

The evaluation of the potential conformations or flexibility in docking is generally challenging, especially in terms of computational power and time.

T-cells utilize clonotypic T cell receptors (TCRs) to recognize antigens and initiate cellular immune responses. TCRs have emerged as a new class of therapeutics, most prominently for the treatment of cancer. Although in some ways similar to antibodies, TCRs differ in the complexity of the receptor-ligand interface: whereas antibodies can be elicited to almost any antigen, TCRs are restricted to antigens presented by MHC proteins. Additionally, TCRs do not undergo affinity maturation, and, similar to naive antibodies, bind with weak-to-moderate affinities and reduced specificity¹.

Recent advances have highlighted the potential therapeutic uses for TCRs with altered binding properties. As T cell potency can be improved with antigen affinity^{2,3}, clinical trials with gene-modified T cells have explored the use of engineered, high affinity TCRs for improved antigen targeting⁴. High affinity TCRs are also used as the antigen targeting component of soluble reagents designed to redirect naive, unmodified T cells⁵.

Multiple methods have been used to generate high affinity TCRs, with the majority created using yeast or phage display (e.g.,^{2,3,6-5}. Recent findings have shown, however, that careful control is necessary when modifying TCRs. Due to their cross-reactive nature, enhancing affinity may introduce new reactivities: improving affinity against one antigen can improve affinity towards others, including those that would otherwise be ignored by the wild-type receptor. This could include self-antigens, leading to possible autoimmune recognition². Such an outcome is believed to have led to fatal off-target reactivity in a recent clinical trial that used a high affinity TCR to target a melanoma antigen⁴. The likelihood of such an outcome is increased if added interaction energy is directed more towards the MHC protein than the peptide. Additionally, the relationship between TCR affinity and potency is not well understood. Although some very high affinity TCRs show considerable sensitivity³, in other cases improving affinity outside an optimal window or above a threshold has led to decreased potency⁹.

Although in vitro evolution has been used to generate the majority of high affinity TCRs, structure-guided computational design offers the potential for finer control over TCR affinity and specificity. Not only can interactions be manipulated in a way that more appropriately address peptide specificity, affinity increments can in principle be more tightly controlled. Towards these goals, structure-guided design has been used to modify a small number of TCRs¹⁰⁻¹³. Recently for example, we used structure-guided design to engineer variants of the DMF5 TCR, which has been used clinically in immunotherapy for melanoma and continues to serve as a model TCR for improving cancer immunotherapy¹¹. Building on an approach originally developed for the well-studied A6 TCR¹⁰, we successfully engineered nanomolar affinity variants of DMF5 with altered specificity, and found excellent agreement between prediction and experiment for both structure and affinity.

Prior approaches used with DMF5 performed poorly with other, unrelated TCRs. This may be attributable to the complexity of TCRs and their interfaces with pMHC, such as varying binding geometries, sub-optimal packing, and differing amounts of receptor and ligand flexibility.

The art of docking and molecular design remains in need of more generalizable approaches such as ways to incorporate flexibility. Such would have significant impact especially in therapeutic molecule design in the medical arts, particularly for those molecules which undergo conformational changes upon binding a target and involve flexibility, such as TCRs, antibodies and enzymes.

### DISCLOSURE OF THE INVENTION

The present invention in a general and overall sense relates to methods and models useful in the design of biomolecules having an identified target.

In one aspect, a computational protein modeling method suitable for selectively modulating an activity of an identified property of a native biomolecule of interest is provided. The method may be used in providing a structure that may be used to provide a modified biomolecule of interest having a selected mutation, such as a deletion, substitution, or other modification. The biomolecule of interest as part of the method will have a known, identified target molecule.

In some embodiments, the method comprising obtaining a set of site-specific RMS fluctuation values for a set of sites of the native biomolecule of interest and for a set of sites of the identified target, incorporating the sets of site-specific RMS fluctuation values into an energy function (sometimes referred to as a force field), computing an interaction energy between a structure or model of the native biomolecule of interest and the identified target using the energy function, computing an interaction energy between a structure or model of the modified biomolecule of interest having a mutation or other modification and the identified target using the energy function, and determining an impact of the mutation or other modification on the binding of the biomolecule to the identified target. In this method, the modulated activity of the modified biomolecule of interest is different compared to the activity of the native biomolecule of interest. The modulated activity may include an increase or decrease of binding affinity, or any other activity being focused for change relative to a native, non-modified form of the biomolecule of interest.

In some embodiments, the mutation of the native, wild-type biomolecule of interest is an amino acid substitution. A particular mutation to be provided to the biomolecule of interest may be selected using structural or computational modeling of the biomolecule according to techniques known to those of skill in the art.

In some embodiments, the biomolecule of interest may comprise a protein, such as a protein of the immune system. By way of example, a protein of the immune system that may be selected is a T-cell receptor (TCR), and a modified T-cell receptor would include a modified amino acid at a site within a CDR1, CDR2 or CDR3 loop of the α or β chain. In some embodiments, the TCR is B7, A6, LC13, DMF4, DMF5, or RD1. In some aspects, the modified TCR will have an enhanced binding affinity for its identified target that is enhanced 10-fold to several hundred fold (400-fold), relative to the binding affinity of the native, wild-type, non-modified TCR. By way of example, the increased affinity of a modified TCR may be described as and increased KD relative to the native TCR, or about 1 μm to about 1 nm.

In some embodiments of the method, the identified target may comprises an antigen associated with a pathogen or cancer. By way of example, the cancer antigen is a melanoma cancer antigen.

In another aspect, a modified biomolecule comprising a non-native, mutated protein is provided. In some embodiments, the mutated protein may comprise several mutations (substitutions, deletions, additions, etc). For example, the mutated protein may be encoded by a non-native amino acid sequence that includes one, two, three or more changes that distinguish the protein from the native, wild—type sequence. The mutated proteins may thus be described as double or triple modified proteins. As demonstrated in the present disclosure, a multiply mutated protein that is synthesized/created/modeled according to the techniques and methods described herein frequently provide an even more greatly enhanced or weakened desired activity that a mutated protein that includes a single mutation.

In another aspect, the invention provides a modified protein that is encoded by a non-native, mutated amino acid sequence, wherein the mutated amino acid sequence includes a selected mutation (deletion, substitution, addition, or other change from native, wild-type) that imparts a modulation of a selected characteristic of the mutated protein changed in some manner from the native, wild type protein. The particular site and/or sites on the amino acid sequence to be modified is selected via assessment of an energy function with a site-specific RMS fluctuation set as described herein. In some embodiments, the modified protein is a modified TCR protein. In particular embodiments, the modified TCR protein comprises an amino acid sequence that includes at least one substitution mutation, the amino acid site or sites having the substitution mutation to be selected via assessment of an energy function incorporating site-specific RMS fluctuations.

In some embodiments, the modified proteins provided according to the present methods are encoded by an amino acid sequence that comprises an amino acid substitution of a small polar or charged amino acid residue with a large hydrophobic or amphipathic amino acid.

An amino acid sequence mutation (change, modification or other) of a modified protein and/or biomolecule of the present invention may be located at any identified site along the sequence encoding the modified protein and/or modified biomolecule. In particular embodiments, especially where the modified protein is a modified TCR protein, the mutation and/or other change to the amino acid sequence will comprise an amino acid substitution at a CDR1, CDR2 or CDR3 loop of the α or β loop.

In yet another aspect, a pharmaceutically acceptable preparation comprising a modified protein provided according to the present methods and models is provided. The preparations may comprise one or more or any combination of such modified proteins. The preparation by include modified proteins alone or together with other, native and/or wild type proteins. The preparation will include a pharmaceutically acceptable carrier, such as a pharmaceutically acceptable carrier of a saline solution or other carrier.

In yet another aspect, a vaccine comprising a pharmaceutically acceptable preparation of at least one modified, non-native, and/or wild-type protein is provided. The modified non-native and/or wild type protein will comprise at least one mutation and/or other change identified and selected according to the present methods.

Definitions:

The following definitions are used in the description of the present invention.

As used in this specification and the appended claims, the singular forms “a,” “an,” and “the” include plural referents unless the content clearly dictates otherwise.

The term “plurality” includes two or more referents unless the content clearly dictates otherwise. Unless defined otherwise, all technical and scientific terms used herein have the same meaning as commonly understood by one of ordinary skill in the art to which the disclosure pertains.

The term “protein” as used herein indicates a polypeptide with a particular secondary and tertiary structure that can participate in, but not limited to, interactions with other biomolecules including other proteins, DNA, RNA, lipids, metabolites, hormones, chemokines, and small molecules.

The term “polypeptide” as used herein indicates an organic polymer composed of two or more amino acid monomers and/or analogs thereof. The term “polypeptide” includes amino acid polymers of any length including full length proteins and peptides, as well as analogs and fragments thereof. A polypeptide of three or more amino acids is typically also called a peptide. As used herein the term “amino acid”, “amino acidic monomer”, or “amino acid residue” refers to any of the twenty naturally occurring amino acids including synthetic amino acids with unnatural side chains and including both D an L optical isomers. The term “amino acid analog” refers to an amino acid in which one or more individual atoms have been replaced, either with a different atom, isotope, or with a different functional group but is otherwise identical to its natural amino acid analog.

The term “small molecule” as used herein indicates an organic compound that is of synthetic or biological origin and that, although might include monomers and/or primary metabolites, is not a polymer. In particular, small molecules can comprise molecules that are not protein or nucleic acids, which play a biological role that is endogenous (e.g. inhibition or activation of a target) or exogenous (e.g. cell signaling), which are used as a tool in molecular biology, or which are suitable as drugs in medicine. Small molecules can also have no relationship to natural biological molecules. Typically, small molecules have a molar mass lower than 1 kgmol.sup. −1. Exemplary small molecules include secondary metabolites (such as actinomicyn-D), certain antiviral drugs (such as amantadine and rimantadine), teratogens and carcinogens (such as phorbol 12-myristate 13-acetate), natural products (such as penicillin, morphine and paclitaxel) and additional molecules identifiable by a skilled person upon reading of the present disclosure.

Experimental structures of proteins in apo and holo (ligand-bound) forms provide snapshots frozen in time, so computational studies of a protein-ligand system and an apo-protein in its physiological environment can provide a rationale for physical forces driving the protein-ligand associations. Insights obtained from such computational studies usually have broader ramifications than just the protein-ligand system of interest. For instance, such insights pertaining to any particular protein-ligand system can be generally utilized in other protein-ligand docking systems and specifically to related protein-ligand docking systems. Similar insights can be obtained for protein-protein systems.

Methods are available for predicting ligand binding sites or strengths (where strengths are often referred to as affinities) in proteins and conformations of ligands interacting with the proteins. However, accurate prediction of ligand binding sites is still a daunting challenge. Any method for prediction of ligand binding sites in proteins will have relevance for many biological applications. For instance, some applications (such as therapeutic applications) can involve design of ligands with desired selectivity and specificity.

Ligand bind site prediction methods generally fall under Virtual ligand screening (VLS) methods and can be categorized into or within two broad areas: a. Structure based prediction of ligand binding modes in proteins, where accuracy of modeling the correct atomic orientations and distances with a particular protein is essential. b. Ligand-based, which is generally used to rank a database of compounds based on similarity to a reference structure. For ligand-based screening, accuracy of proper docked orientations with the protein is generally not essential, but speed of the screening is critical.

Prediction of binding strength is structure based, and is dependent on the accuracy of the atomic orientations and distances, whether derived from experiment (X-ray crystallography, NMR, cryo-EM, etc.) or computer modeling or simulation. This can generally be performed with one of many of available modeling methods, ranging from surface area based empirical functions to more complex energy functions that directly incorporate terms such as electrostatics, van der Waals interactions, hydrophobic solvation, etc. Flexibility is typically ignored, or when incorporated done through expensive and slow computer simulations for each interaction probed.

Prediction methods generally fall within one area or the other. Methods that cover both areas generally are not accurate enough and flexible enough to be applicable to both areas. For instance, many methods that allow for protein flexibility do not provide a standardized implementation to handle protein flexibility. As used in this disclosure, protein flexibility and ligand flexibility refer to physical flexibility of a protein and a ligand, respectively, describing the motions these molecules undergo.

The present disclosure presents a broadly applicable design method that is executed as a computer program aimed at improving a desired characteristic of a biomolecule, such as a protein or peptide, by accurately assessing the flexibility of a biomolecule of interest, and the flexibility of a complex of the biomolecule forms with a ligand, and the flexibility of the ligand, and applying values derived from the flexibility assessments as a general parameters in the design of the biomolecule for achieving a desired modification of an identified activity.

By way of example, and not limitation, the methods provided herein can be used to design a broad range of biomolecules that can be relevant in a number of applications, such as in industrial, biological, enzymatic, pharmaceutical and other applications.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

FIG. 1. Mutations in the interface between the B7 TCR and Tax₁₁₋₁₉/HLA-A2 are scored poorly with the Rosetta interface and ZAFFI 1.1 functions. (a) Structural overview of the B7 TCR-pMHC complex. (b) Score vs. experimental ΔΔG° for point mutations modeled with Rosetta and scored with the Rosetta interface function. The best fit line and correlation coefficient is indicated. (c) As with panel b, scored with the ZAFFI 1.1 function (Haidar et al., 2009; Pierce et al., 2014).

FIG. 2. Experimental ΔΔG° values of TCR point mutations are normal in distribution and affinity-enhancing mutations are overwhelmingly hydrophobic or amphipathic. (a) The 96 point mutations collected in different TCR-pMHC interfaces were approximately normal in distribution with a median ΔΔG° value of 0.5 kcal/mol and a standard deviation of 1.1 kcal/mol. (b) Sequence logos of the 29 mutations that improved binding (ΔΔG°<0).

FIG. 3. Relative TCR-pMHC complex scores correlate better with affinity than binding scores. (a) Scores vs. experimental ΔΔG° for modeled point mutations. Scores were determined by scoring each complex and two free proteins (i.e., binding score=score_{complex}-(score_{TCR}+score_{pMHC})). The wild-type ‘binding score’ was then subtracted from each mutant binding score. After parameterization of Rosetta structural terms, relative binding scores were plotted vs. experimental ΔΔG°. (b) As with panel a, but scores determined and parameterized by scoring only the wild-type and each mutant complex, yielding ‘complex scores’ as described in the text.

FIG. 4. The TR3 score function outperforms our previous TCR design methodology. (a) Complex score vs experimental ΔΔG° for 94 point mutations modeled with Rosetta and scored with the TR3 function. The best fit line, 95% confidence interval, and correlation coefficient is indicated. (b) Performance of our previous methodology applied to the same data. An off-scale prediction score of 26 (DMF5 G28αL) is denoted by a black arrow and the best fit line and correlation are indicated.

FIG. 5. Accounting for buried structural water improves predictions. (a) A buried water molecule observed crystallographically in the DMF5-MART1_{26(27L)-35}/HLA-A2 interface forms multiple electrostatic interactions between the TCR and peptide. The sidechain of Ser99 of the DMF5 β chain is indicated. (b) The correlation between prediction and experiment for models of DMF5 point mutants scored with TR3 is 0.63 when the buried water molecule is ignored. Five mutations at position 99β are indicated and are responsible for the low correlations. (c) The correlation between prediction and experiment for DMF5 point mutants improves to 0.80 when the buried water molecule is treated explicitly. The predicted effects of the five mutations at position 99β agree better with experiment as shown.

FIG. 6. Combining two computationally designed B7 mutations yields nanomolar binding affinity. (a) Combining the S27αM and G99βY mutations in the B7 TCR improves binding to Tax₁₁₋₁₉/HLA-A2 7-fold, from 1.5 μM to 220 nM. (b) The sites of the S27αM and G99βY mutations in the B7 TCR are separated by ˜27 Å and are predicted to improve affinity independently through improved van der Waals interactions with the pMHC.

FIG. 7. Performance of our improved framework on new TCR mutations, HLA-A2 mutations and peptide variations. (a) All point mutation data examined in evaluating our new approach, including TCR, peptide and HLA-A2 data, plotted together, excluding data used in training. The overall correlation between prediction and experiment is 0.86. (b) The predicted effects of MART1_{26(27L)-35} peptide substitutions on the binding of DMF5 to MART1_{26(27L)-35}/HLA-A2 indicate amino acids that are more tolerating of or more sensitive to substitutions. Position 6 near the center of the peptide is particularly sensitive. Each segment of the plot shows the complex scores for all 20 amino acids substituted at the indicated position. Solid lines and numbers in each segment show the average scores for all 20 amino acids at that position. (c) Performance is more limited on a system involving a more diverse, non-HLA-A2 restricted TCR. The impact of mutations in the LC13 TCR with FLR/HLA-B8 are predicted with a correlation coefficient of 0.60 (ΔΔG° values of mutations with no detectable binding were reported previously as 1.6 kcal/mol) (Borg et al., 2005).

FIG. 8. Representative TCR-pMHC SPR binding data for experiments shown in Table I.

TABLE I

| | | TCR mutation | | |
|---|---|---|---|---|
| | | or peptide | ΔΔG° | Error |
| TCR | Peptide^{a} | substitution | (kcal/mol) | (kcal/mol) |
| B7 | Tax | S27αM | −0.43 | 0.08 |
| B7 | Tax | D30αQ | >2 | ND^{b} |
| B7 | Tax | S50αY | −0.73 | 0.09 |
| B7 | Tax | M93αE | >2 | ND |
| B7 | Tax | M93αQ | 1.94 | 0.1 |
| B7 | Tax | Q102αW | 0.56 | 0.14 |
| B7 | Tax | P97βW | >2 | ND |
| B7 | Tax | G98βF | 0.82 | 0.09 |
| B7 | Tax | G99βY | −0.39 | 0.11 |
| B7 | Tax | G99βW | −0.47 | 0.08 |
| B7 | Tax | S27αM/G99βY | −1.15 | 0.1 |
| B7 | Tax | pF3A | 2.7 | 0.02 |
| B7 | Tax | pY5A | 3.28 | 0.11 |
| B7 | Tax | pY5F | 0.55 | 0.04 |
| B7 | Tax | pY8A | 2.76 | 0.07 |
| DMF5 | ELA | D26αF | −0.43 | 0.1 |
| DMF5 | ELA | R27αF | −0.3 | 0.13 |
| DMF5 | ELA | K96αW | −0.65 | 0.12 |
| DMF5 | ELA | T54α1 | 0.33 | 0.12 |
| DMF5 | ELA | S99βF | >2 | ND |
| DMF5 | ELA | S99βH | 1.48 | 0.11 |
| DMF5 | ELA | SS9βI | 1.36 | 0.09 |
| DMF5 | ELA | S99βL | 2.27 | 0.03 |
| DMF5 | ELA | S99βT | 0.4 | 0.13 |
| DMF5 | ELA | pE1A | 0.06 | 0.19 |
| DMF5 | ELA | pE1D | 1.3 | 0.26 |
| DMF5 | ELA | pE1F | 2.26 | 0.06 |
| DMF5 | ELA | PE1Q | 1.0 | 0.03 |
| DMF5 | ELA | pI5E | 3.07 | 0.18 |
| DMF5 | ELA | pG6-Sarc | −0.58 | 0.07 |
| DMF5 | ELA | pL8A | >3 | ND |
| DMF5 | ELA | pT9A | 1.6 | 0.03 |
| DMF5 | ELA | pT9W | >3 | ND |
| DMF4 | ELA | S26αW | −0.63 | 0.04 |
| DMF4 | ELA | T92αW | −0.38 | 0.06 |

FIG. 9. Root mean square fluctuations from MD simulations of free and bound TCRs and pMHC complexes. For TCRs, shaded boxes indicate the locations and values of the six CDR loops. Data for the A6 and B7 TCRs is from Ayres et al., 2016.

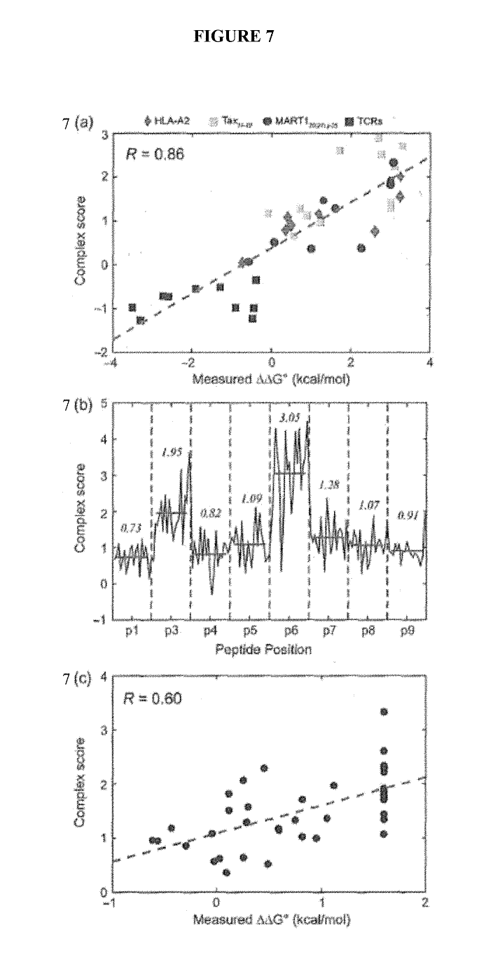

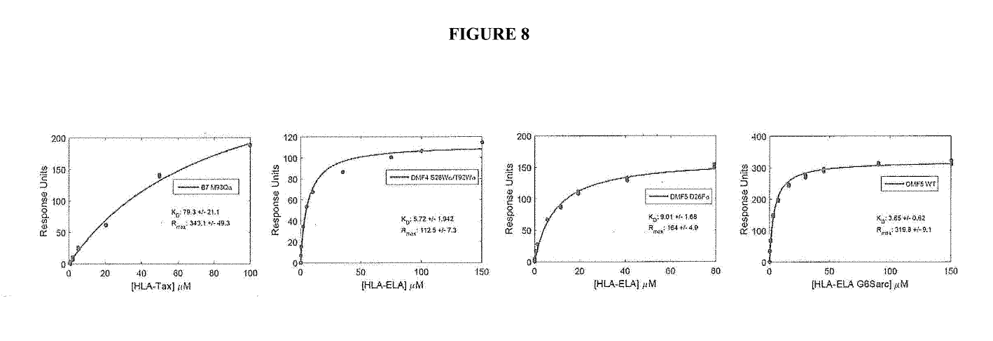

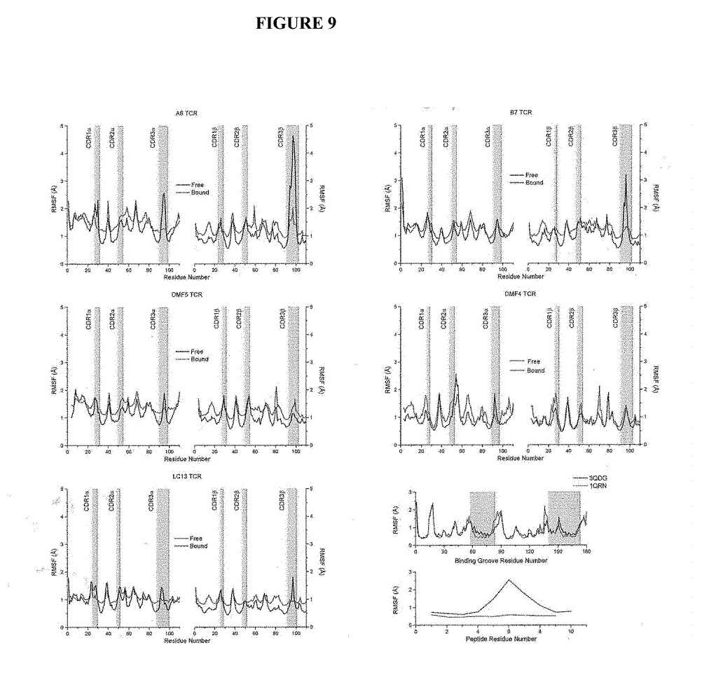

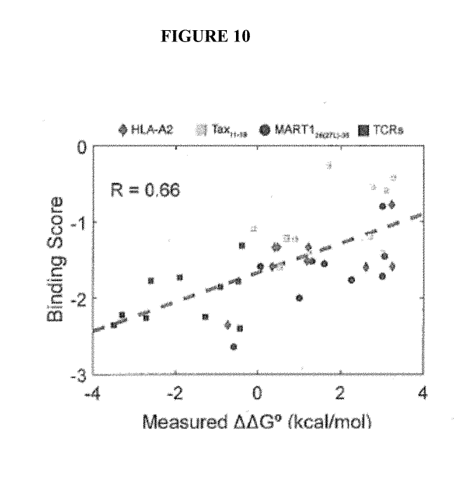

FIG. 10. Performance of our improved framework on new TCR mutations, HLA-A2 mutations, and peptide variations when evaluated using binding rather than complex scores. All point mutation data examined in evaluating our new framework, including TCR, peptide, and HLA-A2 data, are plotted together, excluding data used in training. The overall correlation between prediction and experiment with binding scores is 0.66, compared to 0.86 when using complex scores (compare with FIG. 7a).

FIG. 11. Receiver operating characteristic (ROC) curve for predictions in the LC13 system. The area under the curve is 0.84, indicating good predictive performance when separating affinity increasing mutations from affinity decreasing mutations.

### DETAILED DESCRIPTION OF THE PREFERRED EMBODIMENTS

The present invention provides an improved framework for structure-guided TCR design in a new, general purpose approach to biomolecular design. One example where this is applied is with TCR design. Among other things, the approach employs a novel score function that incorporates flexibility of the receptor (or other biomolecule), ligand and the complex formed between them. Molecular flexibility is specifically and uniquely accounted for in the present design models and methods via a novel cost effective approach. Together with other methodological improvements, a correlation between predicted effect on binding and experimental ΔΔG° of 0.71, compared to 0.16 was achieved. Including all data, the performance of the present method was an impressive 0.79. This is better than seen in a recent large scale analysis of protein design and scoring methods (R=0.64)⁴⁷, but lower than the best upper estimate from the same study (R=0.86). The slope of predicted vs. experimental ΔΔG° was less than 1, indicating that impacts on binding affinity are typically under-estimated.

Accounting for flexibility is an important aspect of the present improved framework, as varying degrees of flexibility is characteristic of biomolecules and their targets. For example, the TCR examples presented here show that the CDR loop, MHC, and peptide flexibility is a characteristic feature of TCRs and pMHC complexes. MD simulations were employed to incorporate flexibility. However, as opposed to simulating structures to identify alternate configurations or generate structural ensembles that had been reported previously²⁵⁻²⁷, using RMS fluctuations as site-specific ‘positional modifiers’ was performed, report on amino-acid level motional properties as terms in the score function. This approach simplifies the treatment of flexibility, requiring only single MD trajectories for the free wild type TCR and the TCR-pMHC complex. Of the properties considered, a-carbon RMS fluctuations were most significant and were incorporated into the final function. The weights for these terms were negative, indicating that more flexible positions are more favorable for design.

The improved framework for TCR design presented here provide for the identification of new affinity-enhancing mutations in multiple interfaces of a biomolecule of interest. For example, methods for modifying and improving target affinity of TCR proteins, such as the DMF4 TCR, which uses different Vα and Vβ genes than those in the training set, are provided. The enhancements to affinity permit a desirable fine control for providing manipulation of TCR affinity. When combined, the predicted mutations provide TCRs having nanomolar binding, as shown for the B7, A6, and DMF5 TCRs.

The present approach accounts for the relative effects of alanine and glycine mutations in TCRs, such as LC13. The present methods may be applied towards other MHC proteins, particularly class II or non-classical MHC proteins. The presently disclosed design approach provides an improved structure-guided biomolecule design, for example, especially in TCR design.

Mutations which enhance TCR affinity include the replacement of small polar or charged residues (such as alanine or glycine) with large hydrophobic or amphipathic amino acids (such as tyrosine or tryptophan. While electrostatic interactions can contribute to specificity, their contributions to affinity can vary due to high desolvation penalties^{56,57}. The present techniques and methods that include a flexibility factor improve the accounting of electrostatic effects, and thus provide a superior means to selectively engineer specificity, thus presenting an improvement that is distinct from conventionally reported methods.

The presently disclosed approach was also able to account for the effects of mutations in the HLA-A2 protein as well as peptide substitutions. Thus, the present computational design may be used for engineering TCRs to modulate their binding properties, and also ligands with enhanced affinity for select TCRs. These approaches may also be employed for peptide-based vaccine design, as well as in the development of new T cell detection or imaging reagents. Additionally, the capacity to accurately score peptide variants according to the present invention provides a method for computationally assessing the cross-reactivity of T cell receptors. This presents the further advantage of predicting and controlling off target toxicity for TCRs used clinically or identifying self-antigens in autoimmunity. By extension, the presently disclosed approach can be used to assess and modulate recognition of other biomolecular-target interactions where cross-reactivity is important.

The following examples present certain embodiments of the invention.

### EXAMPLE 1

### Materials and Methods

The following materials and methods were employed throughout the present examples.

Crystal structure processing and design parameters: For structural modeling, Rosetta with the Talaris2013 score function was used^{16,17,20,21}, using the PyRosetta interface⁶⁰. Native crystal structures were brought to local energy minima through multiple cycles of backbone minimization and rotamer optimization with heavy atom restraints⁶¹. Following structure minimization, the desired TCR, MHC, or peptide mutation was computationally introduced followed by three independent Monte Carlo based simulated annealing trajectories of the TCR CDR loops. This was performed using Rosetta's LoopMover_Refine_CCD mover specified to 3 outer cycles and 10 inner cycles with an initial metropolis acceptance criteria of 2.2 that decreased linearly to 0.6⁶². The large number of resulting packing operations introduced some minor variability when scoring the models. Therefore, the unweighted score terms for the three trajectories were averaged and stored for point mutation energy calculations⁶³. When screening TCR point mutations, TCR residue positions with a center of mass within 10 Å (DMF5 and B7) or 15 Å (DMF4) of a peptide heavy atom were selected for design. For peptide screens, five TCR-facing positions in the MART1_{26(27L)-35} peptide underwent the design procedure. The design process sampled every amino acid (19 mutations and the wild type residue) at each specified position in triplicate. Wild type complexes were modeled and included in scoring to account for impacts of minimization and conformational sampling. For double mutants, both mutations were introduced simultaneously followed by a minimum of six independent minimization trajectories to account for additional structural impacts.

Score function training: To develop a new score function for predicting changes in binding ΔΔG°, Rosetta full atom terms were considered in addition to dynamically derived terms (bound and free order parameters and RMS fluctuations). Multiple linear regression was performed in MATLAB 2015b, using measured ΔΔG° values. A stepwise elimination protocol was used to remove contextually insignificant terms. A k-fold (k=10) cross validation was performed with the data points and significant predictor terms³². The terms and weights for the re-trained energy function are described in Table II.

TABLE II
Terms and their statistics in the TR3 score function

| Term | Weight | Error^{a} | P-value^{b} |
|---|---|---|---|
| Intercept | 2.29 | 0.35 | <0.001 |
| Fa_atr | 0.21 | 0.03 | <0.001 |
| Fa_rep | 0.05 | 0.01 | 0.005 |
| Fa_sol | 0.18 | 0.08 | <0.001 |
| Hbond_sc | 0.34 | 0.09 | 0.008 |
| Rama | 0.12 | 0.05 | 0.119 |
| RMSF_bound | −0.82 | 0.30 | 0.049 |
| RMSF_free | −0.36 | 0.10 | 0.003 |
| Estimated error: 0.81 kcal/mol^{c} | | | |

^{a}Determined as 1.96 standard deviations of k-fold cross-validation weights.
^{b}P-value for the P statistic of the hypotheses test that the corresponding coefficient is equal to zero.
^{c}Average test RMS error from k-fold cross validation.

Modelling explicit water molecules and sarcosine: To model and score buried water molecules and the non-standard sarcosine, explicit TP3 ligands and sarcosine parameters were enabled in Rosetta. Water molecules were placed at their initial crystallographic coordinates followed by 100 high resolution docking trials to coordinate the water molecule in the pocket of the interfaces. The water coordinates were then fixed in position relative to the pMHC for TCR point mutation modelling.

Molecular dynamics simulations of bound and free structures: Molecular dynamics simulations were calculated utilizing the AMBER molecular dynamics suite (Salamon-Ferrer et al, 2013) as described in Ayers et al. 2016. Results for the free and bound A6 and DMF5 were taken from these simulations, with other simulations following the same protocol. Briefly, coordinates for the complexes with the LC13, B7, DMF4 TCRs were obtained from PDB accession codes 1MI5, 1BD2 and 3QDM. Coordinates for the free TAX₁₁₋₁₉/HLA-A2 complex were from 1DUZ. For the LC13, B7, and DMF4 TCRs, coordinates for the free TCRs were obtained by stripping away the pMHC. Prior to simulation, starting systems were charge neutralized with explicit Na+ counterions and solvated with explicit SPC/E water. Following this, systems were energy minimized and heated to 300K with solute restraints. Afterwards, solute restraints were gradually relaxed and followed with 2 ns of simulation with no solute restraints for equilibration, after which 100 ns production trajectories for all systems were calculated. Trajectories were calculated using GPU-accelerated code (Gotz et al, 2012; Salomon-Ferrer et al., 2013). Trajectory analysis including calculation of RMSF values used the ccptraj from the amber suite (Roe and Cheatham 2013). Order parameters were calculated using isotropic reorientational eigenmode dynamic analysis using vectors defined from the Ca to CP (or Ca to H for glycine) atoms (Prompers and Bruschweiler, 2002). For double mutants, descriptors were averaged between the two positions for scoring purposes (i.e., for mutant XY, the RMSF of position Xis averaged with the position Y RMSF to give an RMSF descriptor for XY).

Expression and refolding of soluble constructs of the DMF5, B7, and DMF4 TCRs and HLA-A2 were performed as previously described¹⁵. Briefly, the TCR a and b chains, the HLA-A2 heavy chain, and b₂-microglobulin (b₂m) were generated in Escherichia coli as inclusion bodies, which were isolated and denatured in 8 M urea. TCR a and b chains were diluted in TCR refolding buffer (50 mM Tris (pH 8), 2 mM EDTA, 2.5 M urea, 9.6 mM cysteamine, 5.5 mM cystamine, 0.2 mM PMSF) at a 1:1 ratio. HLA-A2 and b₂m were diluted in MHC refolding buffer (100 mM Tris (pH 8), 2 mM EDTA, 400 mM L-arginine, 6.3 mM cysteamine, 3.7 mM cystamine, 0.2 mM PMSF) at a 1:1 ratio in the presence of excess peptide. TCR and pMHC complexes were incubated for 24 h at 4° C. Afterward, complexes were desalted by dialysis at 4° C. and room temperature respectively, then purified by anion exchange followed by size-exclusion chromatography. Refolded protein absorptions at 280 nm were measured spectroscopically and concentrations determined with appropriate extinction coefficients. Mutations in TCR a and b chains were generated by whole-plasmid mutagenesis and confirmed by sequencing. Peptides were synthesized and purified commercially.

Surface plasmon resonance: Surface plasmon resonance experiments were performed with a Biacore 3000 instrument using CMS sensor chips as previously described¹⁵. In all studies, TCR was immobilized to the sensor chip via standard amine coupling and pMHC complex was injected as analyte. Studies were performed at 25° C. in 20 mM HEPES (pH 7.4), 150 mM NaCl, 0.005% Nonidet P-20. All studies were steady-state experiments measuring RU vs. concentration of injected analyte, and were performed with TCRs coupled onto the sensor chip at 400-2000 response units. Injected pMHC spanned a concentration range of 0.1-150 μM at flow rates of 5 μl/min. Data were processed with BioEvaluation 4.1 and fit using a 1:1 binding model utilizing MATLAB 2015b.

### EXAMPLE 2

### Refinement of the Regression Model to Include Flexibility

Although utilization of complex scores improved the correlation between prediction and experiment, additional predictors of TCR binding affinity that might further improve performance. One of the differences between TCRs is their degree of binding loop flexibility, particularly for the hypervariable CDR3α and CDR3β loops²⁴. Although various methods for conformational sampling such as stochastic loop perturbations or generation of structural ensembles exist²⁵⁻²⁷, these are computationally expensive. To more simply address the impacts of TCR loop flexibility, we considered descriptors from molecular dynamics (MD) simulations of the free and bound TCRs. A comprehensive MD study of the free and bound A6 and DMF5 TCRs (Ayers et al., 2016) using an experimentally bench-marked simulation methodology is described in Scott et al., 2011 and Scott et al., 2012. Similar simulations were performed were performed here on the free and bound B7 TCR. From these simulations root mean square (RMS) fluctuations for each a carbon were determined along with complementary Ca-Cb (Ca-H for glycine) and Ca-C order parameters to quantify backbone flexibility (FIG. 9)

Due to the time that would be required to simulate dozens or hundreds of mutations, only the wild type TCRs and their complexes were simulated. Fluctuation values and order parameters were then treated as ‘positional modifiers’ for each amino acid position, biasing positions for design based on their relative flexibility in the wild type free and bound structures. Although necessary for throughput, this approach makes the limiting assumption that any given mutation does not impact backbone flexibility on the nanosecond timescale.

To determine if inclusion of RMS fluctuations and/or order parameters could lead to an improved TCR scoring function, these six terms were included along with the 16 full-atom Rosetta terms in a multi-linear regression of complex scores vs. ΔΔG°, coupled with a stepwise elimination protocol²⁹. This fit identified six significant (p<0.05) features: four structural terms (van der Waals attractive and repulsive forces, solvation energies, and sidechain hydrogen bonding) and two flexibility terms (RMS fluctuations for a carbons of the free and bound structures). A structural term weighting Ramachandran angle propensities was borderline significant (p=0.11), but was retained in the model to help identify and exclude structural models with residues forced into unrealistic conformations.

The regression models estimated the weights of the RMS fluctuation features to be negative, suggesting flexible positions are more favorable to target for design (although mobility in the complex was weighted more heavily as discussed below). To critically examine the significance of this determination, models with and without the fluctuation terms in addition to the five Rosetta terms were generated and compared. Akaike information criterion (AIC)³⁰ found the incorporation of features describing flexibility resulted in a 99.8% likelihood of a superior prediction model. Bayesian information criterion (BIC)³¹ more strongly penalized additional terms, yet also indicated that inclusion of the fluctuation terms improved the regression model beyond random chance (Table III).

TABLE III
Inclusion of RMS fluctuations improves
the score function regression model

| Criteria | RMSF excluded | RMSF included |
|---|---|---|
| R | 0.63 | 0.71 |
| P-value | 7.9 × 10⁻⁹ | 9.0 × 10⁻¹¹ |
| AIC | 239.2 | 226.8 |
| BIC | 254.4 | 246.2 |

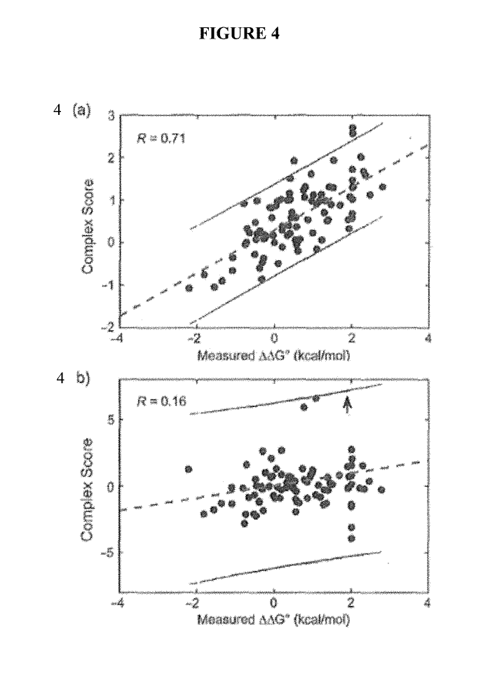

Finally, a k-fold cross validation (k=10)³² was used to validate and estimate overall predictive performance. From this analysis, the RMS error was estimated as 0.81 kcal/mol. After weights for the predictors were chosen, the 94 data points used for training and validation were refit to the regression model, yielding an impressive correlation of 0.71 (FIG. 4a; note this correlation includes accounting for structural water as described below). For comparison, our previous approach with the Rosetta interface score function 11 yielded a correlation of only 0.16 (FIG. 4b). The terms and weights for the final regression model, termed the TR3 score function, are shown in Table II.

### EXAMPLE 3

### Structure Guided Design with the B7 TCR

Based on previous work with the A6 TCR¹⁰, a modeling and scoring scheme was employed to predict the structural and energetic effects of point mutations within interfaces with the ab TCR DMF5. Using this approach, several affinity enhancing mutations in DMF5 were identified that, when combined, led to affinity enhancements towards peptide/MHC of up to 400-fold, compared to the affinity of native peptide.

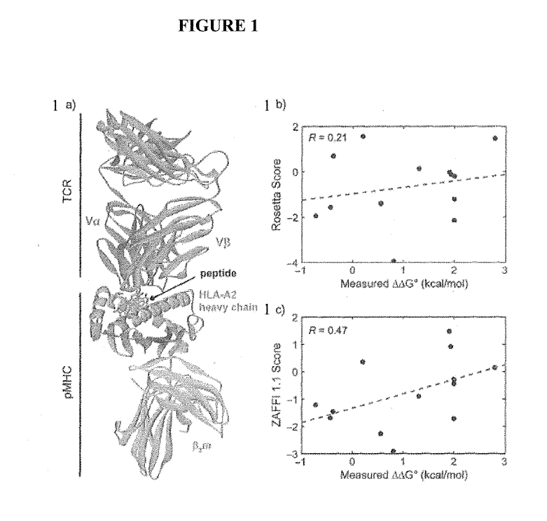

To explore the generality of this approach, B7 TCR was examined. B7 TCR binds the human T-cell lymphoma virus Tax₁₁₋₁₉ peptide presented by HLA-A2 with a similar affinity and orientation as the A6 TCR (FIG. 1a). The A6 and B7 TCRs also share the same germline-derived Vb chain, although crystallographic structures and biophysical studies of A6 and B7 with Tax₁₁₋₁₉/HLA-A2 showed different usage of residues in the binding interface and thermodynamic binding profiles¹⁵. The 740 point mutations in the B7-Tax₁₁₋₁₉/HLA-A2 interface was modelled using Rosetta^{16,17} and the scheme described in Pierce et al¹¹.

Effects were determined by scoring the complex, then separating the components and separately scoring the TCR and pMHC in order to calculate a “binding score”. Based on these scores, nine mutations were selected based on predicted enhancements to binding affinity and chosen for experimental testing.

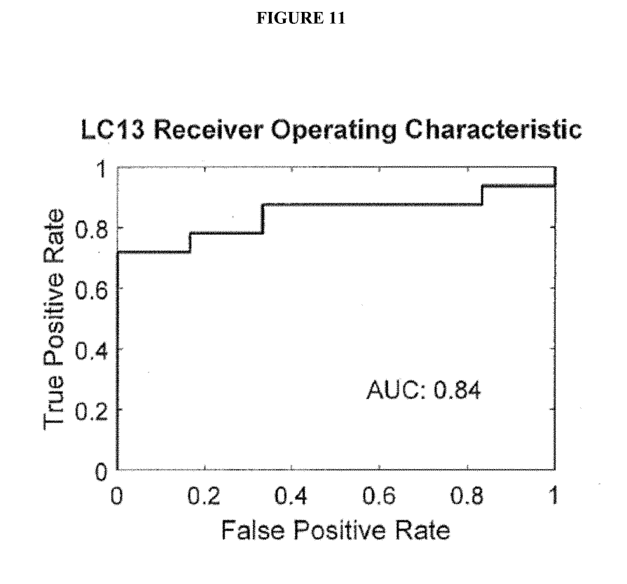

Mutagenesis was performed using soluble B7 gene constructs, expressed and purified the mutant and wild type proteins, and measured their binding affinities toward Tax₁₁₋₁₉/HLA-A2 using surface plasmon resonance (Table II and FIG. 8). Three of the mutations (S27αM, S50αY, G99βY) led to moderately enhanced affinity towards Tax₁₁₋₁₉/HLA-A2, although the remaining six mutations weakened affinity or led to no detectable binding. Including four additional B7 mutations, the correlation between the predicted and experimental change in binding energy was low with the Rosetta interface score function (R=0.21; FIG. 1b). Utilizing the ZAFFI 1 score function developed for the A6 TCR and refined with the DMF5 TCR (Haidar et al., 2009; Pierce et al., 2014), led to an improved but still weak correlation (R=0.47, FIG. 1c). Thus, the TCR design approach developed for the A6 TCR and later applied to DMF5, performs poorly with the B7 TCR.

### Example 4

### Collection of Data Set to Train Computational Predictions in HLA-A2-Restricted TCRs

The present example presents a more generalizable framework for modeling and predicting point mutations across multiple TCR-pMHC interfaces. 96 independent ΔΔG° values were collected that were from single amino acid mutations from four TCR-pMHC interfaces (A6-Tax₁₁₋₁₉/HLA-A2; B7-Tax₁₁₋₁₉/HLA-A2; DMF5-MART1₂₇₋₃₅/HLA-A2; and DMF5-MART1_{26(27L)-35}/HLA-A2). This data originated from other of the inventors structure-guided design efforts with the A6 and DMF5 TCRs (Haider et al., 2009; Pierce et al2014) as well as double mutant cycle deconstruction of the A6 interface (Piepenbrink et al., 2013). Additional data collected with B7 described above was included and new binding measurements in the DMF5-MART1_{26(27L)-35}/HLA-A2 interface (Table II and FIG. 8) were performed. The data set was restricted to high quality measurements with low experimental error (<0.5 kcal/mol).

The point mutations collected in the data set covered a broad range of mutation types as described in Table III.

TABLE III

| | | |
|---|---|---|
| Total mutations in training set | 94 | |
| Polar/charged WT residues | 56 | (60%) |
| Polar/charged mutant residues | 24 | (26%) |
| Mutations with polar/charged WT & mutant residues | 11 | (12%) |
| Large hydrophobic/aromatic WT residues¹ | 14 | (16%) |
| Large hydrophobic/aromatic mutant residue | 41 | (44%) |
| Mutations with large hydrophobic/aromatic WT & mutant | 7 | (7%) |
| residues | | |
| Alanine mutations² | 22 | (23%) |
| Alanine mutations with large/hydrophobic WT residues | 5 | (5%) |

¹Large hydrophobic/aromatic residues defined as Y/W/L/I/F/M
²Excluding mutations with glycine WT residues

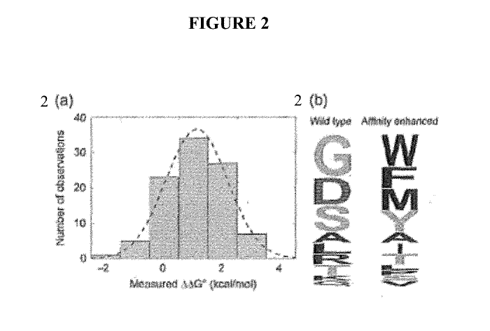

The ΔΔG° values ranged from −1.8 to 2.8 kcal/mol and were approximately normal in distribution (FIG. 2a). The median ΔΔG° value of the selected data set was 0.5 kcal/mol. When comparing the 29 mutations that improved binding, it became evident the majority of affinity-enhancing mutations resulted from replacement of small or polar residues with large hydrophobic residues (FIG. 2b).

### EXAMPLE 5

### Development of a Generalized TCR-pMHC Scoring Function

The present example describes the development of computational structural models of all 96 point mutations for training generalized TCR prediction models. The strategy was extended by adapting techniques for modeling the effects of interface mutations shown to be successful in community-wide assessments. Specifically, the residues were modeled with the standard Talaris2013 score function allowing for off-rotamer sampling and limited backbone flexibility to the CDR loops^{20,21}. Additionally, side chains of residues within a 10 Å sphere of any CDR loop residue were repacked in response to each mutation and resulting CDR loop movements. Each point mutation was modelled in triplicate and scores averaged for further analysis. Analysis of the mutation models identified one with an anomalously high repulsive clash score and another where a residue was forced into an unusual, high energy rotamer. Both of these mutations were excluded from further training and comparisons, leaving a data set of 94 point mutations and their structural models.

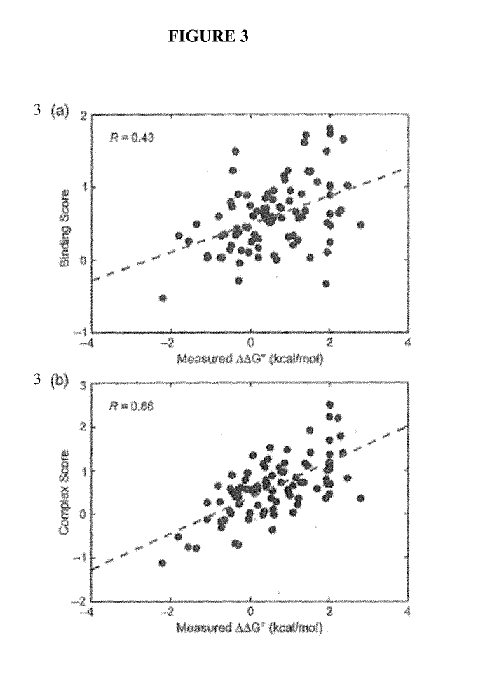

To develop a generalizable TCR scoring function, 16 full-atom Rosetta terms commonly used for protein design and structure prediction^{20,21} were considered. Using the Rosetta terms as predictor variables and experimental binding energies of the data set described above as the response variable, a multi-linear regression was used to parameterize a starting score function for estimating the effect of the various point mutations on ΔΔG°. The most significant contributors to the model (p<0.05) described van der Waals attractive forces and solvation effects. However, the correlation between binding score and ΔΔG° remained low (R=0.43; FIG. 3a). Thus removing insignificant features at this stage was not explored further, in favor of obtaining a more robust prediction model.

Ideally, binding energy calculations would utilize structural information for both the free and bound molecules. However, structures of free TCRs and pMHCs can vary between free and bound states²³, and the large surface areas of receptor and ligand binding sites possess significant conformational degrees of freedom. Relative effects in the present studies were therefore examined by scoring only TCR-pMHC complexes, rather than scoring the complex and the two free proteins as described elsewhere herein. The difference in scores between wild type and mutant complexes are referred to herein as “complex scores.” This approach comes with a limitation in that complex scores do not account for energies in the free TCR associated with making the mutation (i.e., the ΔG for TCR WT↔HTCR mutant). Ideally these would be subtracted when examining the impact of a mutation on binding. There are two potentially significant consequences to this. First, an improved coupling score could arise solely due to improved contacts within the TCR (i.e., better TCR stability). The impact of this impact was minimized by focusing on sites that are in proximity to the ligand and thus more likely to influence binding. Second, any effects on binding stemming from conformational changes in the free TCR will be ignored.

Using the same 16 full-atom Rosetta terms, a multi-linear regression of complex scores vs. ΔΔG° yielded an improved function (R=0.66; FIG. 3b). Despite the theoretical limitations noted above, complex scores are therefore more applicable for the present framework and were used for all further calculations. The improvement using complex scores may reveal underlying limitations in the energy function terms and/or limitations in recapitulating conformational differences between free and bound TCR's as noted above, leading to inacurracies when “binding scores” are computed. The inherently weak affinities and correspondingly poor quality of TCR-pMHC interfaces (compared to e.g., to high affinity antibody-antigen interfaces) could also contribute to why complex scores outperform binding scores.

Therefore, complex scores are more applicable in the present platform and were used for all further calculations.

### Example 6

### Accounting for Energetically Significant Structural Water Improves Predictions

Rosetta utilizes a Lazaridis-Karplus implicit solvation model to estimate solvation energies associated with bulk water³³. However, TCR-pMHC interfaces are large and buried water molecules are often observed crystallographically. In some instances these structural waters play key roles in the interface that would not be captured with an implicit solvation model³⁴.

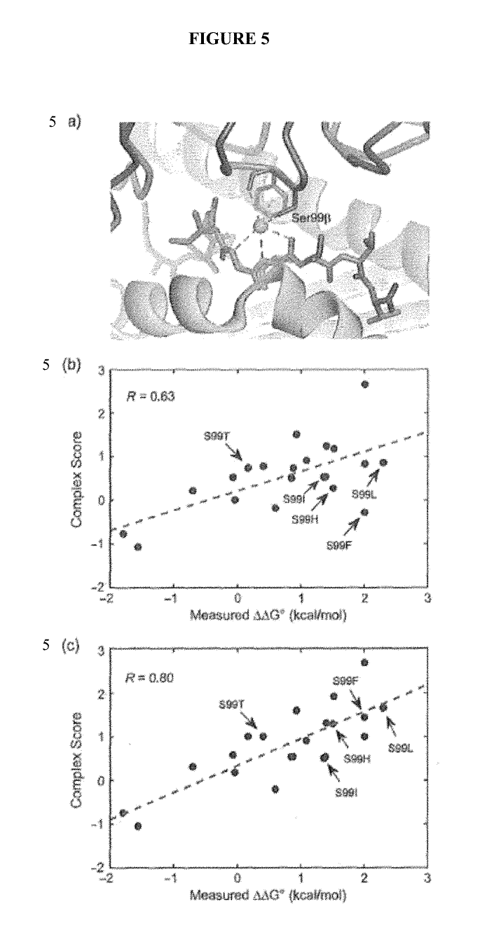

Many predicted mutations which filled the void of an interfacial water molecule in the interface with the DMF5 TCR resulted in a falsely favorable score. For example, Ser99 in the DMF5 b chain contacts the peptide, but is also involved in a complex water-mediated hydrogen bond network linking the peptide to the TCR (FIG. 5a). The predicted impacts of mutations at this position did not correlate well with experiment (FIG. 5b), consistent with a determination that this water molecule is structurally and energetically significant. To directly account for it, the buried water in the DMF5 interface was docked into its corresponding pocket and treated explicitly in modeling and scoring. This improved the agreement between prediction and experiment for Ser99b point mutations without altering the predictions for distant residues (FIG. 5c). Further design studies incorporated this technique when buried water molecules were observed crystallographically in the interface between peptide and TCR (i.e., in the DMF5 and LC13 TCRs as described herein).

### EXAMPLE 7

### Validation with New TCRs Mutations and Combinations to Modulate Affinity

The present example demonstrates the applicability of the new modeling framework on mutations defined herein to screen for new mutations in the interfaces with the DMF5 and B7 TCRs (DMF5-MART1_{26(27L)-35}/HLA-A2and B7-Tax₁₁₋₁₉/HLA-A2). To emphasize peptide specificity, only positions with a center of mass within 10 Å of a peptide heavy atom were selected for design. A total of 18 sites in both DMF5 and B7 were modeled and scored using TR3 with all 20 amino acids (684 point mutations in total and 36 wild type controls). Most mutations were predicted to have deleterious effects on binding. However, several mutations were predicted to enhance affinity. Some of these were at sites where mutations had been reported to favorably impact binding (Table II).

The two predicted to be most favorable (G99bW for B7; D26aF for DMF5) were both generated, and the impact on binding assessed experimentally. Both mutations improved binding as was predicted using the herein disclosed model. The ΔΔG° for G99bW in B7 was −0.5 kcal/mol; for D26aF in DMF5 it was −0.4 kcal/mol. The value for D26aF was less than observed previously with tyrosine or tryptophan at this position (−1.6 and −1.8 kcal/mol, respectively), suggesting that the amphipathic character of tyrosine and tryptophan may be advantageous for enhancing TCR affinity as discussed below.

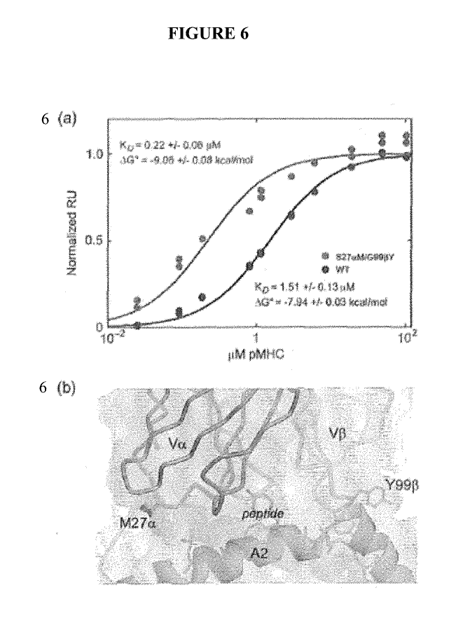

Previous designs for the A6 and DMF5 TCRs combined multiple mutations to generate molecules which bound in the nanomolar range^{10,11}. The approximate additive effects of mutations in both interfaces were captured by the presently disclosed new framework with the TR3 score function after averaging the RMSF positional values of each of the mutations. To demonstrate if the new framework also allowed for this in another TCR, the S27aM and G99bY mutations were combined in the B7 receptor, which together improved the B7 affinity for Tax₁₁₋₁₉/HLA-A2 seven-fold, from 1.5 μM to 200 nM (FIG. 6). These mutations are approximately 27 Å apart, and were correctly predicted to be additive when combined (=−1.2 kcal/mol, complex score ΔΔG°=−0.77).

To investigate the broader applicability of the TR3 score function, mutations in an additional TCR not included in training were modeled and scored with TR3. The DMF4 TCR also recognizes MART1 antigens presented by HLA-A2, but utilizes different a and 13 chains than DMF5, A6, and B7^{35,36}. As performed with the A6, B7, and DMF5 TCRs, MD simulations of the free wild-type DMF4 TCR and its complex with MART1_{26(27L)-35}/HLA-A2 were performed and used along with Rosetta to simulate 960 structures (19 mutations at 48 sites, and 48 wild type controls) in the DMF4-MART1_{26(27L)-35}/HLA-A2 interface. Several mutations in the α chain were favorably ranked based on their ability to fill an interfacial void near the N-terminus of the peptide. Three of these mutations were selected for experimental investigation (S26aW, N29aW, and T92aW). Although the N29aW mutation was of particular interest as it provided another opportunity to investigate a structural water, this mutant could not be folded from inclusion bodies. This left two mutations for experimental testing. Both of these enhanced DMF4 binding affinity, with ΔΔG° values of −0.4 and −0.6 kcal/mol (Table 51). These mutations were also found to be additive when combined: together the S26αW and T92αW mutations and improved the affinity of the DMF4, TCR 10-fold, from 60_to 6 μM (ΔΔG° of −1.4 kcal/mol).

Overall, when applied to the data outside of the training set, the new modeling and scoring procedure disclosed here recapitulated the effects of multiple mutations in the B7, DMF5 and DMF4 TCRs, and permitted the identification of new affinity-enhancing mutations in all three receptors. The RMS error between predicted and experimentally determined impacts on binding was 1.5 kcal/mol, higher than observed with training and cross-validation but still lower than observed with the previous methodology used (FIG. 7a, black points).

### EXAMPLE 8

### Improved Framework Predicts the Outcome of HLA-A2 Mutations

ab TCRs show MHC restriction, i.e., they recognize peptides only when presented by MHC proteins (Zinkernagel and Doherty, 1974). Some reports examine the effects of mutations in the a helices of MHC binding groove as a means to determine energetically significant positions that might guide restriction, including a recent comprehensive analysis of the binding of A6 TCR to the Tax₁₁₋₁₉/HLA-A2 complex¹⁹.

Of nine published mutations, eight weakened affinity and one enhanced affinity. To recapitulate this data in silico, the impact of mutations in HLA-A2 on the binding of A6 to Tax₁₁₋₁₉/HLA-A2 was modeled, incorporating free and bound flexibility through molecular dynamics simulations as described above. The effects of these mutations were well captured, with RMS error between prediction and experiment of 1.0 kcal/mol (FIG. 7a, green points). The new framework is therefore applicable to TCRs, and can predict the energies associated with mutations in the HLA-A2 side of the interface as well.

### EXAMPLE 8

### Computational Scanning of Peptide Variants

TCRs are broadly cross reactive and recognize a multitude of antigenic peptides, a requirement of the fixed size of the T cell repertoire³⁷. Additionally, altering TCR binding by changing peptide sequence is another approach for modulating TCR binding and immune responses^{38,39}. Quantitative data for how eight substitutions in the TAX11-19 peptide impact the binding of the A6 TCR is available, and new alanine scanning data for recognition of four more TAX11-19 variants by B7 was therefore collected.

As with the HLA-A2 mutations, the new modeling and scoring approach described herein was used to assess how these peptide variants impact recognition by A6 and B7. The impacts on binding ΔΔG° were recapitulated well, within an RMS error of 0.9 kcal/mol (FIG. 7a, yellow points).

To further demonstrate the utility of the present approach for assessing peptide variations, residues in the MASRT126(271.)-35 peptide were computationally varied to cover all 20 amno acids, and after completing a MD simulation of the MART1_{26(27L)-35}/HLA-A2 complex, scored for impact on DMF5 binding. All peptide substitutions were predicted to be unfavorable, although mutations at the P3 and P6 positions were predicted to have the most dramatic impacts (FIG. 7b). This outcome is consistent with recent findings on TCR specificity, which suggest the existence of peptide ‘hotspots’ of reduced structural and chemical diversity, outside of which greater variation is permitted (Adams et al., 2016).

Next, eight MART1_{26(27L)-35} peptide variants with a broad range of complex scores were selected for experimental testing with DMF5. A peptide was also examined with a non-standard sarcosine (N-methyl glycine) substituted for Gly6 of the peptide to help test the implications of treating structured water explicitly in the DMF5 interface as discussed above and shown in FIG. 5. Overall, there was a good correlation between ΔΔG° and binding score for the nine MART1_{26(27L)-35} peptide variants explored experimentally, with experiment and prediction differing with an RMS error of 0.9 kcal/mol (FIG. 7a, blue points). The experiments with the sarcosine-modified peptide led to improved binding as predicted, leading to a 3-fold affinity enhancement in affinity (ΔΔG° of −0.6 kcal/mol). The affinity enhancement is attributable to the increased van der Waals interactions to Thr102 of the TCR while maintaining the solvated state of polar atoms in the surrounding pocket.

Thus, structure guided design incorporating flexibility via site-specific positional modifies determined from RMS fluctuations may be used in assessing the impact of peptide variations.

### EXAMPLE 9

### Overall Performance and Exploration of an Even More Diverse, Non HLA-A2 Interface

To explore the overall performance of our new approach, we examined the new TCR mutations, HLA-A2 mutations and peptide variants described above together as one large test set. These amounted to 40 independent ΔΔG° measurements distinct from the training set from five different TCR-pMHC interfaces. We also included the double mutants in the DMF5, B7 and A6 TCRs. Altogether, performance was excellent, with predicted and experimental impacts on binding agreeing with an impressive correlation coefficient of 0.86 and a RMS error of 1.1 kcal/mol, spanning a large range of −7 kcal/mol in binding free energy (FIG. 7a, all points). Complex scores again showed improved performance over binding scores, as scoring the 40 test set mutations using binding scores yielded a weaker correlation coefficient (R=0.66) and large RMS error 92.8 kcal/mol) (FIG. S3).

The systems used in development and testing involved the class I MHC protein HLA-A2. The systems were used to assess the impact of mutations between the interface of the LC13 TCR and the class I MHC protein HLA-B:08:01 (HLA-B8) presenting the FLR peptide (sequence FLRGRAYGL).

The structure of the LC13-FLR/HLA-B8 complex has been determined, as have ΔΔG° values for 39 alanine or glycine mutations in the various LC13 CDR loops (Borg et al., 2005. After completing MD simulations of LC13 and its complex, the approach described herein was applied to this dataset, recapitulating the effects of these mutations with an overall correlation of 0.60 and an RMS error of 1.0 kcal/mol (FIG. 7c). While errors are still within the range obtained with our previous methodology (Pierce et al., 2014), the correlation is weaker than what was achieved with HLA-A2 restricted systems.

While not intending to be limited to any particular theory or mechanism of action, the weaker performance with the LC13 TCR may be related to several factors. First, many of the 39 mutations in the LC13 interface result in very weak or no detectable binding, with ΔΔG° values reported simply as above an upper limit of 1.6 kcal/mol (corresponding to a 15-fold weakening of affinity). The limited accuracy of these measurements will affect the correlation between prediction and experiment. As evidence of this, binary metrics demonstrated good predictive performance when separating affinity increasing mutations from affinity decreasing mutations (ROC AUC=0.84; FIG. S4). Second, the HLA-A2 restricted systems in parameterization of the new TR3 score function could result in an inherent bias. HLA-A2 and HLA-B8 differ by 42 amino acids, 32 of which are in the peptide binding domain (Robinson et al., 2011). In addition to different energetic contributions, these differences could alter the structural and dynamic responses to mutations in ways that less well specifically captured in some embodiments of the present methods.

### EXAMPLE 10

### Design Method with Industrial Enzymes, DNA, and other Biomolecules

The present example is provided to demonstrate the utility of the presently described molecular design models and methods for use in creation of a wide scope of biomolecules, including enzymes, DNA, and other molecules.

The following steps may be used in the creation and/or modeling of a desired biomolecule product.

1. Identify the desired design goal (e.g., property of the biomolecule desired to be changed) for the biomolecule of interest and its interaction with an identified target.
a. Modulate (improve or weaken) affinity of antibody towards antigen
b. Modulate (improve or weaken) affinity of TCR towards antigen/MHC complex
c. Modulate (improve or weaken) affinity of enzyme towards substrate or inhibitor
d. Modulate (improve or weaken) affinity of transcription factor towards DNA
e. Modulate (improve or weaken) affinity of receptor antagonist towards receptor
f. Modulate specificity of any biomolecular interaction

2. Perform molecular dynamics (MD) simulations on the “wild type” (i.e native, unmodified) biomolecule, target, and complex. Input coordinates for molecular dynamics simulations can come from experimental structures (X-ray, NMR, cryo-EM, etc.) or computational models.

3. From the molecular dynamics simulations, calculate RMS fluctuations for each site in the native, unmodified biomolecule, target, and complex. A site may include:
a. An amino acid site, using RMS fluctuations for individual atoms (individual backbone atoms or averages thereof, side chain atoms or averages thereof, amino acid centers of mass, etc.)
b. A monomer in a nucleic acid (atoms of bases or averages thereof, atoms of nucleic acid sugar-phosphate backbones or averages thereof, base/sugar-phosphate centers of mass, etc.)

4. Incorporate terms reflecting RMS fluctuations for a site to be examined into an energy function relating molecular structure to energy (referred to as a “force field”). Force fields are used by software packages such as CHARMM, AMBER, Rosetta or variants/derivatives thereof.

5. Model mutations or chemical variations at each site of interest in the biomolecule in its free and in its bound form using a modeling protocol. Modeling protocols that may be used include:
a. Simple modeling using structural investigation and chemical intuition
b. Computational modeling tools such as those employed by commercial or publicly available software (Rosetta, Discovery Studio, etc.) that incorporate conformational sampling with various forms of molecular mechanics, molecular dynamics, and/or energy minimization

6. Using the modified energy function incorporating RMS fluctuation terms generated in step 4 and the models generated in step 5, calculate the effect of the mutation/modification on binding of the biomolecule using approaches used in molecular modeling/protein design, to include:
a. Calculating the energy of the complex of the mutated/modified biomolecule and its target, the energy of the wild-type biomolecule and its target, then taking the difference (interaction score).
b. Calculating the energy of the complex of the mutated/modified biomolecule and its target, the energy of the mutated/modified biomolecule alone, the energy of the target alone, then subtracting the energy of the mutated/modified biomolecule and the energy of the target from that of the complex; performing the same set of calculations with the wild-type biomolecule, then taking the difference between the two sets of calculations (binding score).

### EXAMPLE 11

### Application to the Therapeutic TCR DMF4

The present example is provided to demonstrate the utility of the presently described molecular design models and methods for use in modification of the therapeutic DMF4 TCR to improve specificity for the melanoma associated MART1 peptide.

The following steps may be used in the modeling and creation of a specific variant of the DMF4 TCR.

1. Perform molecular dynamics (MD) simulations on the “wild type” (i.e native, unmodified) DMF4 TCR, MART1 peptide:MHC molecule, and complex. Input coordinates for molecular dynamics simulations can come from available experimental structures.

2. From the molecular dynamics simulations, calculate RMS fluctuations for each amino acid alpha carbon in the native, unmodified TCR, pMHC, and complex.

4. Incorporate RMS fluctuation values for each site to be examined in the DMF4 TCR into the Rosetta energy function/force field.

5. Model mutations of the 20 standard amino acids at each site of interest in the DMF4 TCR in its free and in its bound form using the Rosetta modeling protocol.

6. Calculate, using the modified energy function generated in step 4, the energy of the complex of the mutated/modified DMF4 TCR and the pMHC, the energy of the wild-type TCR and the pMHC, then determine the difference.

7. Rank the energy calculated for each mutation, and select the lowest energy mutations predicted to engage the peptide or higher energy mutations predicted to engage the MHC for experimental analysis. Mutations may be in any of the CDR1, CDR2, or CDR3 loops of the α or β chain.

8. Make the lowest energy mutations and test experimentally for enhanced binding.

9. Repeat step 8 for the next lowest energy mutation and test; repeat again as needed to identify one or more affinity-enhancing mutations.

10. Combine affinity-enhancing mutations identified in steps 8-9 to generate modified DMF4 TCR with desired affinity.

### EXAMPLE 12

### Application to a Therapeutic Antibody

The present example is provided to demonstrate the utility of the presently described molecular design models and methods for use in modification of a therapeutic antibody to improve recognition of the immune checkpoint receptor PD-1.

The following steps may be used in the modeling and creation of a high affinity variant of the nivolumab antibody.

1. Perform molecular dynamics (MD) simulations on the unmodified nivolumab antibody, PD-1 receptor, and complex. Input coordinates for molecular dynamics simulations can come from available experimental structures or computational models.

2. From the molecular dynamics simulations, calculate RMS fluctuations for each amino acid center of mass in the unmodified antibody, PD-1, and complex.

4. Incorporate RMS fluctuation values for each site to be examined in the nivolumab antibody into the Rosetta energy function/force field.

5. Model mutations of the 20 standard amino acids at each site of interest in the nivolumab antibody in its free and in its bound form using the Rosetta modeling protocol.

6. Calculate, using the modified energy function generated in step 4, the energy of the complex of the mutated/modified antibody and its target, the energy of the unmodified antibody and its target, then determine the difference.

7. Rank the energy calculated for each mutation, and select the lowest energy mutations for experimental analysis.

8. Make the lowest energy mutations and test experimentally for enhanced binding.

9. Repeat step 8 for the next lowest energy mutation and test; repeat again as needed to identify one or more affinity-enhancing mutations.

10. Combine affinity-enhancing mutations identified in steps 8-9 to generate modified antibody with desired affinity.

### EXAMPLE 13

### Application to an Enzyme of Interest

The present example is provided to demonstrate the utility of the presently described molecular design models and methods for use in modification of a peptide inhibitor to inhibit activity of the Caspase-3 enzyme.

The following steps may be used in the modeling and creation of a high affinity peptide to inhibit Caspase-3 activity.

1. Perform molecular dynamics (MD) simulations on the unmodified Caspase-3 enzyme, peptide, and complex. Input coordinates for molecular dynamics simulations can come from available experimental structures or computational models.

2. From the molecular dynamics simulations, calculate RMS fluctuations for each amino acid carbonyl in the native, unmodified enzyme, peptide, and complex.

4. Incorporate RMS fluctuation values for each site to be examined in the Caspase-3 enzyme into the CHARM force field.

5. Model mutations of the 20 standard amino acids at each site of interest in the peptide in its free and in its bound form using the Discovery Studio modeling protocol.

6. Calculate, using the modified force field generated in step 4, the energy of the complex of the modified peptide and Caspase-3, the energy of the modified peptide alone, the energy of the Caspase-3 alone, then subtracting the energy of the modified peptide and the energy of the Caspase-3 from that of the complex.

7. Rank the energy calculated for each peptide predicted, and select the lowest energy peptides for experimental analysis.

8. Make the lowest energy mutations and test experimentally for enhanced binding.

9. Repeat step 8 for the next lowest energy mutation and test; repeat again as needed to identify one or more affinity-enhancing mutations.

10. Combine affinity-enhancing mutations identified in steps 8-9 to generate modified enzyme with desired affinity.

The examples set forth above are provided to give those of ordinary skill in the art a complete disclosure and description of how to make and use the embodiments of the methods for prediction of the selected modifications that may be made to a biomolecule of interest, and are not intended to limit the scope of what the inventors regard as the scope of the disclosure. Modifications of the above-described modes for carrying out the disclosure can be used by persons of skill in the art, and are intended to be within the scope of the following claims.

It is to be understood that the disclosure is not limited to particular methods or systems, which can, of course, vary. It is also to be understood that the terminology used herein is for the purpose of describing particular embodiments only, and is not intended to be limiting. A number of embodiments of the disclosure have been described. Nevertheless, it will be understood that various modifications may be made without departing from the spirit and scope of the present disclosure. Accordingly, other embodiments are within the scope of the following claims.

### BIBLIOGRAPHY

The following references are specifically incorporated herein in their entirety
1. Baker, B. M., Scott, D. R., Blevins, S. J. & Hawse, W. F. Immunological Reviews 250, 10-31 (2012).
2. Zhao, Y. et al. Journal of Immunology 179, 5845-5854 (2007).
3. Varela-Rohena, A. et al. Nat Med (2008).
4. Linette, G. P. et al. Blood 122, 863-871 (2013).
5. Oates, J. & Jakobsen, B. K. Oncolmmunology 2, e22891 (2013).
6. Li, Y. et al. Nat Biotech 23, 349-354 (2005).
7. Holler, P. D. et al. Proc Natl Acad Sci USA 97, 5387-5392 (2000).
8. Bowerman, N. A. et al. Mol Immunol 46, 3000-3008 (2009).
9. Stone, J. D. & Kranz, D. Frontiers in Immunology 4 (2013).
10. Haidar, J. N. et al. Proteins: Structure, Function, and Bioinformatics 74, 948-960 (2009).
11. Pierce, B. G. et al. PLoS Comput Biol 10, e1003478 (2014).
12. Zoete, V., Irving, M., Ferber, M., Cuendet, M. & Michielin, 0. Frontiers in Immunology 4 (2013).
13. Malecek, K. et al. Specific Increase in Potency via Structure-Based Design of a TCR. The Journal of Immunology 193, 2587-2599 (2014).
14. Ding, Y. H. et al. Immunity 8, 403-411 (1998).
15. Davis-Harrison, R. L., Armstrong, K. M. & Baker, B. M. Journal ofMolecular Biology 346, 533-550 (2005).
16. Kaufmann, K. W., Lemmon, G. H., DeLuca, S. L., Sheehan, J. H. & Meiler, J. Biochemistry 49, 2987-2998, doi:10.1021/bi902153g (2010).
17. Das, R. & Baker, D. Annu Rev Biochem 77, 363-382 (2008).
18. Kortemme, T. & Baker, D. Proceedings of the National Academy of Sciences of the United States of America 99, 14116-14121 (2002).
19. Piepenbrink, K. H., Blevins, S. J., Scott, D. R. & Baker, B. M. Nat Commun 4 (2013).
20. Leaver-Fay, A. et al. in Methods in Enzymology Vol. 523 (ed E. Keating Amy) 109-143 (Academic Press, 2013).
21. Moretti, R. et al. Proteins: Structure, Function, and Bioinformatics 81, 1980-1987 (2013).
22. Vreven, T., Hwang, H., Pierce, B. G. & Weng, Z. Protein Science 21, 396-404, doi:10.1002/pro.2027 (2012).
23. Armstrong, K. M., Piepenbrink, K. H. & Baker, B. M. Biochem J415, 183-196.
24. Scott, D. R., Borbulevych, O. Y., Piepenbrink, K. H., Corcelli, S. A. & Baker, B. M. Journal of Molecular Biology 414, 385-400 (2011).
25. Tuffery, P. & Derreumaux, P. Journal of The Royal Society Interface 9, 20-33 (2012).
26. Sinko, W., Lindert, S. & McCammon, J. A. Chemical Biology & Drug Design 81, 41-49 (2013).
27. Feixas, F., Lindert, S., Sinko, W. & McCammon, J. A. Biophysical Chemistry 186, 31-45, (2014).
28. Scott, Daniel R., Vardeman Ii, Charles F., Corcelli, Steven A. & Baker, Brian M. Biophysical Journal 103, 2532-2540 (2012).
29. Hocking, R. R. A Biometrics Invited Paper. Biometrics 32, 1-49, (1976).
30. Akaike, H. in Selected Papers of Hirotugu Akaike (eds Emanuel Parzen, Kunio Tanabe, & Genshiro Kitagawa) 215-222 (Springer New York, 1998).
31. Kass, R. E. & Raftery, A. E. Bayes Factors. Journal of the American Statistical Association 90, 773-795 (1995).
32. Arlot, S. & Celisse, A. A survey of cross-validation procedures for model selection. 40-79 (2010).
33. Lazaridis, T. & Karplus, M. Proteins: Structure, Function, and Bioinformatics 35, 133-152 (1999).
34. Jiang, L., Kuhlman, B., Kortemme, T. & Baker, D. Proteins: Structure, Function, and Bioinformatics 58, 893-904 (2005).
35. Johnson, L. A. et al,. Blood 114, 535-546 (2009).
35. Borbulevych, 0. Y., Santhanagopolan, S. M., Hossain, M. & Baker, B. M. J Immunol187, 2453-2463 (2011).
36. Mason, D. Immunology Today 19, 395-404 (1998).
37. Piepenbrink, K. H. et al. Biochemical Journal 423 (2009).
38. McMahan, R. H. & Slansky, J. E. Seminars in Cancer Biology 17, 317-329 (2007).
39. Borg, N. A. et al. Nat. Immunol. 6, 171-180 (2005).
40. Robinson, J. et al. The IMGT/HLA database. Nucleic acids research 39, D1171-1176 (2011).
41. Restifo, N. P., Dudley, M. E. & Rosenberg, S. A., Nat Rev Immunol 12, 269-281 (2012).
42. Morgan, R. A. et al. Journal of Immunotherapy 36, 133-151 (2013).
43. Parkhurst, M. R. et al. Mol Ther 19, 620-626, (2011)
44. Borbulevych, O. Y. et al. Immunity 31, 885-896 (2009).
45. Insaidoo, F. K. et al. J Biol Chem 286, 40163-40173 (2011).
46. Potapov, V., Cohen, M. & Schreiber, G. Protein Engineering Design and Selection 22, 553-560 (2009).
47. Haidar, J. N. et al. Journal of Molecular Biology 426, 1583-1599, (2014).
48. Shoemaker, B. A., Portman, J. J. & Wolynes, P. G. Proceedings of the National Academy of Sciences 97, 8868-8873 (2000).
49. Hawse, W. F. et al. The Journal of Immunology 192, 2885-2891 (2014).
50. Janin, J. Structure 7, R277-R279, (1999).
51. Rodier, F., Bahadur, R. P., Chakrabarti, P. & Janin, J. Proteins: Structure, Function, and Bioinformatics 60, 36-45 (2005).
52. de Graaf, C., Pospisil, P., Pos, W., Folkers, G. & Vermeulen, Journal of Medicinal Chemistry 48, 2308-2318 (2005).
53. Bui, H.-H., Schiewe, A. J. & Haworth, I. S. WATGEN: Journal of Computational Chemistry 28, 2241-2251 (2007).
54. Koide, S. & Sidhu, S. S. ACS Chemical Biology 4, 325-334 (2009).
55. Bosshard, H. R., Marti, D. N. & Jelesarov, I. J. Mol Recognit 17, 1-16 (2004).
56. Hendsch, Z. S. & Tidor, B. Protein Sci 3, 211-226 (1994).
57. Procko, E. et al. Journal of Molecular Biology 425, 3563-3575, (2013).
58. Collis, A. V. J., Brouwer, A. P. & Martin, A. Journal of Molecular Biology 325, 337-354, (2003).
59. Chaudhury, S., Lyskov, S. & Gray, J. J. PyRosetta: Bioinformatics 26, 689-691 (2010).
60. Bradley, P., Misura, K. M. S. & Baker, D. Science 309, 1868-1871 (2005).
61. Canutescu, A. A. & Dunbrack, R. L. Protein Science: A Publication of the Protein Society 12, 963-972 (2003).
62. Kellogg, E. H., Leaver-Fay, A. & Baker, D. Proteins: Structure, Function, and Bioinformatics 79, 830-838 (2011).

1. A computational protein modeling method suitable for selectively modulating an activity of an identified property of a native biomolecule of interest to provide a modified biomolecule of interest having a selected mutation, said biomolecule of interest having an identified target, the method comprising:
obtaining a set of site-specific RMS fluctuation values for a set of sites of the native biomolecule of interest and for a set of sites of the identified target;
incorporating the sets of site-specific RMS fluctuation values into an energy function;
computing an interaction energy between a structure or model of the native biomolecule of interest and the identified target using the energy function;
computing an interaction energy between a structure or model of the modified biomolecule of interest having a mutation or other modification and the identified target using the energy function; and
determining an impact of the mutation or other modification on the binding of the biomolecule to the identified target,
wherein the modulated activity of the modified biomolecule of interest is different compared to the activity of the native biomolecule of interest.

2. The method of claim 1 wherein the mutation is an amino acid substitution identified with structural or computational modeling.

3. The method of claim 2 wherein the biomolecule is a protein.

4. The method of claim 3 wherein the protein is an immune system protein and the modified protein is a modified immune system protein.

5. The method of claim 4 wherein the immune system protein is a T-cell receptor (TCR) and the modified immune system protein is a modified T-cell receptor that comprises a modified amino acid at a site within a CDR1, CDR2 or CDR3 loop of an α or β chain.

6. The method of claim 3 wherein the identified target is an antigen associated with a pathogen or cancer.

7. The method of claim 5 wherein the TCR is B7, A6, LC13, DMF4, DMF5, or RD1.

8. The method of claim 6 wherein the cancer antigen is a melanoma cancer antigen.

9. The method of claim 5 wherein the modified T-cell receptor has a KD for a target protein of about 1 μM to about 1 nM.

10. The method of claim 3 wherein the protein is mutated to provide a double or triple modified protein.

11. (canceled)

12. (canceled)

13. (canceled)

14. (canceled)

15. (canceled)

16. (canceled)
